(19) United States
(12) Patent Application Publication
(10) Pub. No.: US 2022/0088261 A1
(43) Pub. Date: Mar. 24, 2022

(54) SANITIZING SYSTEM FOR VEHICLE COMPARTMENT

(71) Applicant: Bergstrom, Inc., Rockford, IL (US)

(72) Inventors: Terry Zeigler, Byron, IL (US); Brett S. Connell, Winnebago, IL (US); Ross Koehler, Winnebago, IL (US)

(21) Appl. No.: 17/458,123

(22) Filed: Aug. 26, 2021

Related U.S. Application Data
(60) Provisional application No. 63/081,092, filed on Sep. 21, 2020.

(51) Int. Cl. A61L 9/20; A61L 2/10

(52) U.S. Cl. CPC A61L 9/20; A61L 2/10; A61L 2202/11; A61L 2209/14; A61L 2202/25; A61L 2209/11; A61L 2202/14

(57) ABSTRACT

The various embodiments described herein include methods, devices, and systems for sanitizing a vehicle. In one aspect, a sanitizing system includes a sanitizing system housing configured to be disposed within a compartment of the vehicle, an air filtration assembly, and an ultraviolet lighting assembly. The air filtration assembly includes an air inlet and an air outlet in fluid communication with the compartment of the vehicle, an air filter, and a blower fan. The ultraviolet lighting assembly includes a UV light source, and a reflector disposed between the UV light source and the sanitizing system housing. The reflector is configured to direct UV light from the UV light source into the compartment of the vehicle.

### RELATED APPLICATIONS

This application claims priority to U.S. Provisional Patent Application No. 63/081,092, filed Sep. 21, 2020 which is hereby incorporated by reference in its entirety.

### TECHNICAL FIELD

The systems and methods described herein relate generally to sanitizing systems, including but not limited to, systems for sanitizing an interior compartment of a vehicle.

### BACKGROUND

During the use and/or storage of public and private vehicles, the vehicles' interior compartments may be exposed to or otherwise contaminated with pathogens, such as bacteria, viruses, and other microorganisms that can cause disease. Vehicle compartments can be sanitized to prevent occupants from being exposed to pathogens within the vehicle compartment. Vehicles can be sanitized on a regular interval or between periods of vehicle service. Chemical-based sanitizing operations can be costly, time-consuming, and expose cleaning crews to the pathogens and harmful sanitizing chemicals, and expose later users of the vehicle to the harmful chemicals. Finally, the sanitizing chemicals may also cause degradation of the materials within the compartment, like plastics, seat fabrics, etc.

### SUMMARY

Accordingly, there is a need for systems and/or devices with more efficient and effective methods for sanitizing vehicle compartments. Such systems, devices, and methods optionally complement or replace conventional systems, devices, and methods for sanitizing vehicle compartments.

The present disclosure describes chemical-free sanitizing systems to provide sterile or otherwise sanitized vehicle compartments. In some embodiments, the sanitizing system includes a system housing that is configured to be disposed within the compartment of the vehicle. The housing can be temporarily or permanently mounted to the vehicle. The housing can be mounted to the ceiling of the vehicle compartment. In some embodiments, the sanitizing system can include a service assembly configured to be introduced into a vehicle compartment.

In various embodiments, the sanitizing system includes an air filtration assembly. The air filtration assembly can include an air inlet, an air outlet, an air filter, and a blower fan. In some embodiments, the air filtration assembly is integrated with the lighting assembly of the sanitizing system. In some embodiments, the air filtration assembly is disposed within a service assembly housing.

In some embodiments, the sanitizing system includes an ultraviolet lighting assembly. The ultraviolet lighting assembly can include a UV light source and a reflector. In various embodiments, the UV light source is configured to emit light with a wavelength of approximately 100 nanometers to approximately 280 nanometers. In some embodiments, the UV light source comprises a halogen light bulb, a florescent light bulb, or an LED light bulb. In some embodiments, the lighting assembly includes a plurality of UV light sources.

In some embodiments, the reflector is configured to emit UV light at an angle spanning between approximately 120 to 160 degrees. In various embodiments, the reflector includes a semi-circular cross-sectional profile. The reflector can include a tab to permit an operator to rotate the reflector. In various embodiments, the reflector is movable with an actuator, like a servo-motor or the like.

In some embodiments, the sanitizing system further includes a controller. The controller can be configured to control emittance of UV light emitted by the UV lighting assembly within the compartment of the vehicle, and filter air by the filtration assembly within the compartment of the vehicle.

In some embodiments, the controller is configured to simultaneously: (i) control emittance of UV light, and (ii) filter air by the filtration assembly. In various embodiments the controller is further configured to control rotation of the reflector to control dosing or exposure of ultraviolet light emitted by the lighting assembly within the compartment of the vehicle.

In some embodiments, the sanitizing system includes a remote control wirelessly coupled to the controller, wherein the remote control is configured to remotely activate and deactivate the sanitizing system.

In various embodiments, the sanitizing system includes a display operatively coupled to the controller, wherein the display is configured to display an emittance strength and/or duration of UV light by the lighting assembly within the compartment of the vehicle, and display an exchange of air by the filtration assembly within the compartment of the vehicle.

In some embodiments, the sanitizing system includes a timer configured to start the system and thereafter turn off the system after a predetermined period of time set by an operator (or turn off at a designated time). In some embodiments, the sanitizing system also introduces ozone into the compartment.

In some embodiments the controller is operatively coupled to the vehicle. In various embodiments, the controller is further configured to control operation of the sanitizing system when the vehicle is in an engine off state. In some embodiments, the controller is further configured to control operation of the sanitizing system when the vehicle is unoccupied.

In some embodiments, the UV bulb fitting is disposed above the air outlet. In some embodiments, the air outlet is disposed above the air inlet. In some embodiments, the air outlet comprises an elongate shape. In some embodiments, the UV bulb fitting and the reflector each comprise an elongate shape. In some embodiments, the air inlet is in fluid communication with outside the vehicle.

In various embodiments, the sanitizing system includes an air ionizer in fluid communication with the air inlet and the air outlet.

As discussed previously, conventional chemical-based sanitizing operations can be costly, time-consuming, and can expose personnel or users to pathogens and potentially harmful chemicals. The present disclosure includes embodiments that allow for chemical-free sanitization within minimal human intervention. The sanitization system can include an air filtration assembly to filter and/or purify air during the sanitization process. The sanitization system can further include an air ionizer and/or an air conditioner to further treat the air within the vehicle compartment. The sanitization system can also include a heating element to further sterilize surfaces within the vehicle compartment by raising the temperature of the compartment to above a temperature that most viruses and bacteria can survive. The sanitization system can be integrated into the vehicle to allow for frequent or regular sanitization of the vehicle compartment. Further, by integrating the sanitization system with the vehicle, the vehicle compartment can be automatically sanitized based on time of use, scheduling, and/or time of day. The sanitization system can also be provided as a service tool that is easily introduced into a vehicle for sanitization of the vehicle compartment. This is particularly useful for rental vehicles and/or people movers (e.g. buses) that are used by many different individuals over a short amount of time.

In one aspect, some embodiments include a sanitizing system housing configured to be disposed within the compartment of the vehicle; an air filtration assembly disposed at least partially within the housing, said air filtration assembly including an air inlet configured to be disposed in fluid communication with a compartment of a vehicle; an air outlet configured to be in fluid communication with the compartment of the vehicle; an air filter disposed between the air inlet and the air outlet; and a blower fan configured to direct air from the air inlet to the air outlet through the filter; and an ultraviolet (UV) lighting assembly including a UV bulb fitting configured to receive a UV bulb; and a reflector disposed between the UV bulb and a portion of the sanitizing system housing, the reflector configured to direct UV light from the UV bulb into the compartment of the vehicle.

In another aspect, some embodiments include a service assembly including: a service housing configured to be disposed within the compartment of the vehicle; an air inlet defined in the service housing, the air inlet in fluid communication with the compartment of the vehicle; an air outlet defined in the service housing, the air outlet in fluid communication with the compartment of the vehicle; a filter disposed between the air inlet and the air outlet; and a blower disposed within the service housing and configured to direct air from the air inlet to the air outlet through the filter; and at least one lighting assembly operatively coupled to the service assembly, the at least one lighting assembly including: a lighting assembly housing; a light bulb coupled to the lighting assembly housing, the light bulb configured to emit ultraviolet light when energized; and a reflector disposed between the light bulb and the lighting assembly housing, the reflector defining a beam angle to direct the ultraviolet light into the compartment of the vehicle.

In another aspect, some embodiments include a sanitizing system housing configured to be disposed within the compartment of the vehicle; an air filtration means for filtering air within a compartment of a vehicle, the air filtration means at least partially disposed within the sanitizing system housing; and a lighting means for directing ultraviolet (UV) light into the compartment of the vehicle.

In another aspect, some embodiments include a method including directing air from within the compartment of the vehicle through a filter via an air inlet; reintroducing the air into the compartment of the vehicle after passing through the filter via an air outlet; dosing the compartment of the vehicle with ultraviolet light via a light bulb; and directing the ultraviolet light within the compartment of the vehicle along a desired beam angle via a reflector; and rotating the reflector relative to the light bulb to control dosing of the compartment of the vehicle with ultraviolet light.

In another aspect, some embodiments include a configured to perform any of the methods described herein. In another aspect, some embodiments include a non-transitory computer-readable storage medium storing one or more programs, the one or more programs comprising instructions which, when executed by a system, cause the system to perform any of the methods described herein.

Thus, systems and devices are provided with more efficient and effective methods for sanitizing vehicle compartments, thereby increasing the effectiveness, efficiency, and safety with such systems and devices.

### BRIEF DESCRIPTION OF THE DRAWINGS

For a better understanding of the various described embodiments, reference should be made to the Description of Embodiments below, in conjunction with the following drawings in which like reference numerals refer to corresponding parts throughout the figures.

FIG. 1 is an elevation view of a vehicle in accordance with some embodiments.

FIG. 2 is an elevation view of a vehicle in accordance with other embodiments.

FIG. 3 is an elevation view of a vehicle in accordance with yet other embodiments.

FIG. 4 is a perspective view of a representative system in accordance with some embodiments.

FIG. 5 is a bottom view of the system of FIG. 4.

FIG. 6 is a partial cross-sectional view of a representative lighting assembly in accordance with some embodiments.

FIG. 7 is a partial perspective view of the lighting assembly of FIG. 6 in accordance with some embodiments.

FIG. 8 is a partial perspective view of the lighting assembly of FIG. 6 in accordance with other embodiments.

FIG. 9 is a perspective view of a representative system in accordance with some embodiments.

FIG. 10 is a perspective view of a representative lighting assembly in accordance with other embodiments.

FIG. 11 is a perspective view of a representative lighting assembly in accordance with yet other embodiments.

FIG. 12 is a perspective view of a representative service assembly in accordance with further embodiments.

FIG. 13 is a perspective view of a representative display in accordance with still further embodiments.

### DETAILED DESCRIPTION

The present disclosure describes various embodiments of chemical-free sanitizing systems for vehicles. In some embodiments, a sanitizing system is integrated or otherwise coupled to the vehicle. For example, a vehicle can include a sanitizing system that is attached to the ceiling or floor of the vehicle. In this example, lighting assemblies disposed on the ceiling of the vehicle can emit UV light onto surfaces of the vehicle compartment to sanitize the vehicle compartment. In some embodiments, the sanitizing system can include an air filtration assembly to filter or purify air within the vehicle cabin. In some embodiments, the sanitizing system includes an air ionizer, an air conditioner, and/or heating elements. In another example, the sanitizing system is a service tool that can be temporarily placed in a vehicle. In this example, the sanitizing system is battery powered.

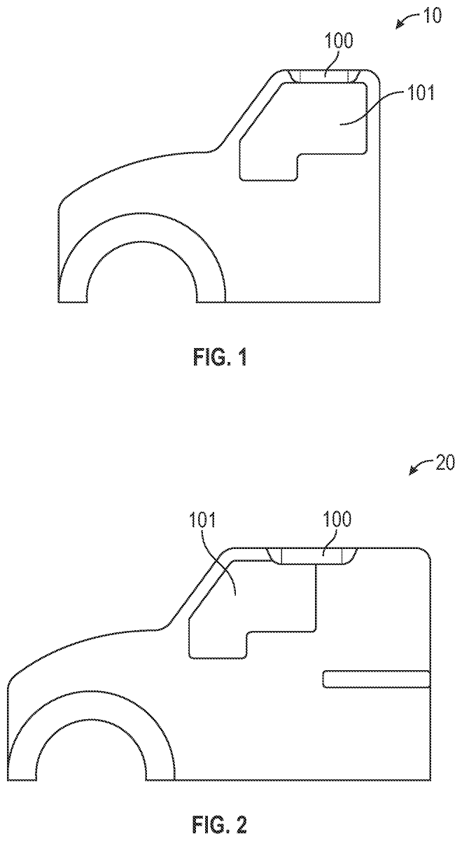

FIG. 1 is an elevation view of a vehicle 10 in accordance with some embodiments. In the depicted example, the vehicle 10 can be any suitable vehicle, including vehicles for public use and/or vehicles for private use. The vehicle 10 can be a truck or tractor utilized for commercial purposes. During operation and/or storage of the vehicle 10, the vehicle compartment 101 can be exposed to or otherwise contaminated with pathogens such as bacteria, viruses, and other microorganisms that can cause disease.

As described herein, a sanitizing system 100 disposed within the vehicle compartment 101 can sanitize the vehicle compartment 101 without the use of chemicals (other than in some embodiments, Ozone). In the depicted example, the sanitizing system 100 can expose surfaces of the vehicle compartment 101 to radiation, such as ultraviolet (UV) light, to disinfect the surfaces of the vehicle compartment 101 by inactivating pathogens on surfaces of the vehicle compartment 101.

In some embodiments, the sanitizing system 100 can be permanently mounted or disposed within the vehicle compartment 101, such that the sanitizing system 100 remains in the vehicle compartment 101 at all times unless it needs to be removed for repair or replacement. In other embodiments, the sanitizing system 100 can be semi-permanently or temporarily mounted or otherwise disposed within the vehicle compartment 101. Optionally, the sanitizing system 100 can be mounted to the ceiling of the vehicle compartment 101.

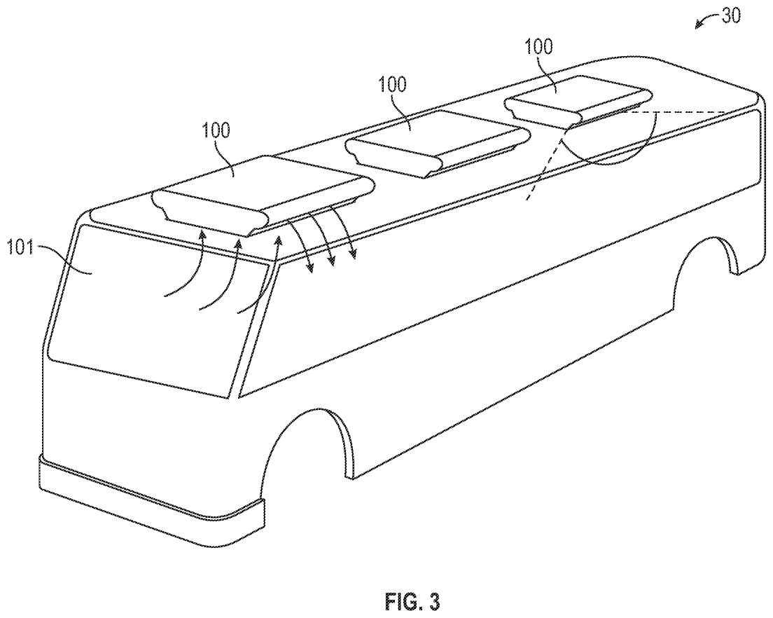

FIG. 2 is an elevation view of a vehicle 20 in accordance with some embodiments. In the depicted example, the sanitizing system 100 can be utilized with long-haul trucks or tractors 20 to sanitize the vehicle compartment 101 of the vehicle 20. FIG. 3 is an elevation view of a vehicle 30 in accordance with some embodiments. As can be appreciated, the sanitizing system 100 can be utilized with coaches 30 that may serve as private transit, city transit, school buses, etc. Optionally, multiple sanitizing systems 100 can be utilized in a larger vehicle compartment 101 of a coach 30. As illustrated, multiple sanitizing systems 100 can be disposed along the length of the vehicle compartment 101. The sanitizing systems 100 can be mounted to the ceiling of the vehicle compartment 101 to maximize passenger space and comfort. In some embodiments, sanitizing systems 100 can be mounted to the floor of the vehicle compartment 101. Optionally, sanitizing systems 100 can be mounted to the floor between or underneath seating or storage compartments within the vehicle compartment 101.

While several examples are described herein, sanitizing systems can be attached to or otherwise used with any suitable vehicle, including, but not limited to cruise ships, train cars, automobiles, police cars, prisoner transports, cargo areas, etc.

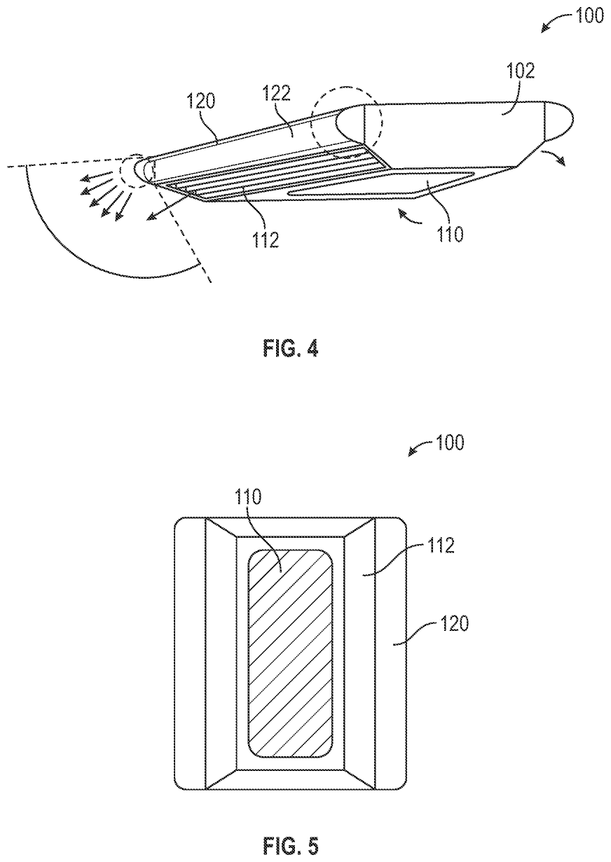

FIG. 4 is a perspective view of a representative system 100 in accordance with some embodiments. FIG. 5 is a bottom view of the system 100 of FIG. 4 in accordance with some embodiments. With reference to FIGS. 4 and 5, the sanitizing system 100 can sanitize the vehicle compartment by exposing surfaces within the vehicle compartment to radiation, such as UV light, to disinfect the vehicle compartment.

In the depicted example, the system housing 102 can be temporarily or permanently disposed within the vehicle. In some applications, the system housing 102 can be mounted, temporarily or permanently, to the vehicle. Optionally, the system housing 102 can be configured and/or can include features to mount to the ceiling of a vehicle compartment. Advantageously, by ceiling mounting the system housing 102, the sanitizing system 100 can maximize passenger space while allowing for effective placement of UV lights, air filtration components, and other components of the sanitizing system 100.

As described herein, the sanitizing system 100 can include varying levels of integration with the vehicle. In some embodiments, the sanitizing system 100 can be powered by the vehicle. Functions and operation of the sanitizing system 100 may also be controlled by controls or controllers within the vehicle.

In the depicted example, the sanitizing system 100 includes one or more light assemblies 120 to direct radiation, such as UV light, to surfaces within the vehicle compartment to sanitize the vehicle compartment. In some embodiments, the sanitizing system 100 can include one light assembly 120, two light assemblies 120, or four light assemblies 120. The light assemblies 120 can be disposed along the length or sides of the system housing 102. The light assemblies 120 can be mounted to the system housing 102. Optionally, the light assemblies 120 can be integrated or otherwise monolithic with the system housing 102. The light assemblies 120 can be detachable or removable from the system housing 102 for positioning within the vehicle compartment.

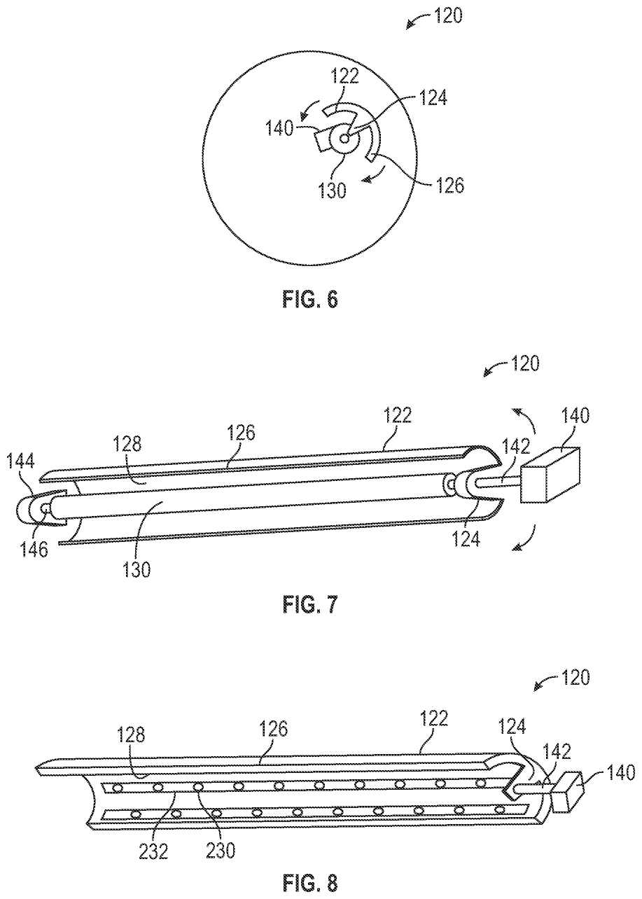

FIG. 6 is a partial cross-sectional view of a representative lighting assembly 120 in accordance with some embodiments. FIG. 7 is a partial perspective view of the lighting assembly 120 of FIG. 6 in accordance with some embodiments. With reference to FIGS. 4-7, each light assembly 120 can include a bulb or light source 130 in a fitting that radiates UV light. In some embodiments, the light source 130 can be a halogen bulb that is configured to emit UV light, or a florescent bulb configured to emit UV light. The light sources 130 can have a generally elongate shape and may extend along the length of the system housing 102.

The light source 130 can radiate any suitable type of radiation or light that may be use for sanitizing or otherwise inactivating pathogens, including, bacteria and/or viruses. Light emitted by the light source 130 can be in the ultraviolet C (UV-C) range, having a wavelength between 100 to 280 nanometers (nm). Advantageously, UV-C light can provide a germicidal effect or action, inactivating or breaking down DNA of microorganisms, preventing the replication of pathogens. Therefore, the use of UV-C light exposure within the vehicle compartment can effectively sterilize the vehicle compartment with respect to viruses, bacteria, molds, spores, etc.

In the depicted example, the light assembly 120 can include a reflector 122 to direct the light emitted by the light source 130 toward surfaces in the vehicle compartment. The reflector 122 can be disposed between the light source 130 and the system housing 102. In some applications, the reflector 122 can be configured to direct a light beam from the light source 130 approximately 120 degrees to 160 degrees, allowing wide coverage of the surfaces within the vehicle compartment. The reflector body 126 can have a reflector surface 128 that is covered or coated with a light reflective material or can have a surface finish that reflects light from the light source 130.

As illustrated, the reflector 122 can have a generally semi-circular cross-sectional profile. A reflector body 126 can extend from a central tab 124 of the reflector 122. The reflector body 126 can generally have a curved profile. The reflector body 126 can have a parabolic shape. In the depicted example, the reflector body 126 can extend in a generally elongate shape and may extend along the length of the system housing 102. The profile and/or shape of the reflector body 126 can be configured or adjusted based on applications as well as properties or characteristics of the light source 130.

As described herein, the reflector 122 can be movable or otherwise configurable to allow the user and/or the sanitizing system 100 to direct or distribute the light beam from the light source 130 toward desired portions of the vehicle compartment. In some applications, a user can manually move or rotate the reflector 122 to direct the light beam to desired surfaces within the vehicle compartment. As can be appreciated, by directing the light beam to desired portions of the vehicle compartment, the dosing of UV-C light on various surfaces can be controlled. Optionally, the central tab 124 can be utilized to pivot, rotate, or otherwise move the reflector 122 relative to the light source 130.

Advantageously, by allowing the reflectors 122 to be adjusted or otherwise manipulated, the lighting assembly 120 can allow for a greater range of light coverage (in excess of 180 degrees to 270 degrees of exposure) compared to conventional lighting assemblies with fixed reflectors (which may offer approximately 60 degrees to 90 degrees of exposure). By allowing for increased exposure, the light assemblies 120 can be mounted in a variety of locations while allowing for desired levels of exposure or dosing of UV-C light. Optionally, the lighting assemblies 120 can be mounted in high positions (e. g. ceiling mount), low positions (e. g. floor mount), or other positions (along or on doors or windows, etc.).

In some embodiments, the lighting assembly 120 can include an actuator 140 to move or change the position of the reflector 122 without user intervention. During operation, the actuator 140 can sweep or otherwise move the reflector 122 in a pattern to expose desired area of the vehicle compartment to UV-C light. Advantageously, by sweeping the light beam of the light source 130, fewer lighting assemblies 120 can be utilized while providing a desired dosing of UV-C light.

In some embodiments, an actuator shaft 142 of the actuator 140 can couple to the central tab 124 to allow the actuator 140 to rotate the reflector 122. Optionally, the lighting assembly 120 can include a secondary actuator 144. The secondary actuator 144 can similarly include an actuator shaft 146 coupled to the central tab 124 on an opposite side to allow the secondary actuator 144 to rotate the reflector 122. The actuator 140 and the secondary actuator 144 can cooperatively rotate the reflector 122. In some embodiments, the secondary actuator 144 may be a support member to allow the reflector 122 to pivot or rotate.

Optionally, the sanitizing system 100 can control the position of the reflector 122 to control the dosing or exposure of various portions of the vehicle compartment to ensure all desired surfaces are sanitized. The actuator 140 and/or the secondary actuator 144 can be controlled to determine the rotational speed and/or position of the reflector 122, and therefore the light beam from the light source 130. In some applications, the rotational speed or position of the reflector 122 can be determined based on user input or integrated controls. As can be appreciated, by controlling the rotation of the reflector 122, the dosing of UV-C light within the vehicle compartment can be controlled to allow for desired levels of sterilization in various portions of the vehicle compartment.

FIG. 8 is a partial perspective view of the lighting assembly 120 of FIG. 6 in accordance with some embodiments. In the depicted example, the lighting assembly 120 can utilize LED light sources 230. The LED light sources 230 can include a diffuser 232 to spread light coverage from multiple point light sources 230. In some applications, the lighting assembly 120 can utilize a modified or different reflector 122 to direct light from LED light sources 230.

Referring back to FIGS. 4 and 5, optionally, the sanitizing system 100 can filter, purify, or exchange air within the vehicle compartment. The sanitizing system 100 can include an air filtration assembly to filter air drawn in from the air inlet 110 and directed into the vehicle compartment through the air outlet 112. In some embodiments, the air filtration assembly is integrated within the system housing 102.

During operation, a blower can draw air into the air filtration assembly through the air inlet 110. The blower can be an AC powered blower or a DC powered blower.

The air inlet 110 can be formed through a portion of the system housing 102. As illustrated, the air inlet 110 can be positioned at a lower portion of the system housing 102. The air inlet 110 can have a generally rectangular shape, and may be sized to occupy a lower panel of the system housing 102. Optionally, the air inlet 110 can be disposed below the lighting assemblies 120 of the system housing 102. Further, the air inlet 110 can be centralized between the lighting assemblies 120. As can be appreciated, the air inlets 110 can be direct fluid communication with the vehicle compartment of the vehicle. In some embodiments, the air inlets 110 can be configured to draw in air from the environment surrounding the vehicle to allow fresh airflow to enter the vehicle compartment.

In the depicted example, air drawn into the air filtration assembly can pass through a filter to remove contaminants from the air flow. The filter can be any suitable filter, including, but not limited to high efficiency particulate air (HEPA) filters.

After filtration, the air can be directed into the vehicle compartment through one or more air outlets 112. The air outlets 112 can be formed through a portion of the system housing 102. The air outlets 112 can have a generally elongate shape and may extend along the length of the lighting assemblies 120. The air outlets 112 can be positioned at a lower portion of the system housing 102. As illustrated, the air outlets 112 can be disposed between the lighting assemblies 120 and the air inlet 110. Optionally, the air outlets 112 can be configured to direct air flow across the light emitted by the lighting assemblies 120, sterilizing air flow exiting the sanitizing system 100.

As can be appreciated, the air outlets 112 can be direct fluid communication with the vehicle compartment of the vehicle. In some embodiments, the air outlets 112 can be configured to direct air from the vehicle compartment into the environment, expelling air from within the vehicle compartment.

In some embodiments, the air filtration assembly can include an air ionizer to further purify or remove airborne bacteria and/or viruses from the vehicle compartment. Optionally, the air filtration assembly can include air conditioning components to allow air flow into the vehicle compartment to be conditioned.

Optionally, the sanitizing system 100 can utilize heat to sanitize the vehicle compartment. The sanitizing system 100 can include one or more heating elements to heat the vehicle compartment. In some applications, the heating elements can raise the temperature of the vehicle compartment in excess of 133 degrees to sanitize or otherwise inactivate pathogens within the vehicle compartment. As can be appreciated, the heating elements can be utilized as a compliment to light-based sterilization to allow sterilization of surfaces or areas where light from the sanitizing system 100 may not reach.

In the depicted example, a controller can control the operation of the sanitizing system 100. For example, a controller can be configured to operate the lighting assemblies 120 to sterilize a vehicle compartment. The controller can further be configured to operate other components of the sanitizing system 100, such as the air filtration assembly, the air ionizer, the air conditioner, and/or the heating element. In some applications, the controller can be configured to allow the lighting assemblies 120 and the air filtration assembly to operate simultaneously, allowing for simultaneous sterilization and air filtration of a vehicle compartment. The controller can be configured to allow simultaneous and/or sequential operation of the components or assemblies described herein.

The controller can control the power to the lighting assemblies, and therefore the emittance of radiation, such as UV-C light, emitted from the lighting assemblies 120. Further, the controller can control the blower motor speed of the air filtration device to control the amount of air flow or air exchanged though the air filtration assembly. Similarly, the controller can control operational parameters of the air ionizer, the air conditioner, and/or the heating element.

In some embodiments, the controller can allow for the sanitizing system 100, or components thereof, to operate for a desired or pre-determined period of time. The sanitizing system 100 can include a timer to allow the lighting assemblies 120, the air filtration assembly, the air ionizer, the air conditioner, and/or the heating element to operate for a desired period of time. The sanitizing system 100 can include a timer interface to allow a user to select a period of time to operate the sanitizing system 100.

In some applications, the controller can be in communication with, or integrated with the vehicle, or vehicle controllers. Advantageously, the controller can operate the sanitizing system 100 in accordance with a determination of the vehicle state of operation. For example, the sanitizing system 100 can be configured to operate when the vehicle is in an engine-off state. Further, the sanitizing system 100 can be configured to operated when a vehicle senses that no occupants are within the vehicle compartment. In some embodiments, a determination of vehicle occupancy can be made by information received by a motion detector or other occupancy sensor.

Optionally, the controller can be configured to operate the sanitizing system based on vehicle and occupant parameters. For example, the controller can be configured to operate the sanitizing system 100 based on a determination that the engine is off and no occupants are within the vehicle compartment. Further, the controller can be configured to operate the sanitizing system 100 based on the time of day or night, or if the vehicle has been operation for a predetermined amount of time. As an example, the controller can be configured to operate the sanitizing system 100 between rentals or shifts.

In some applications, the sanitizing system 100 can be operated remotely. The sanitizing system 100 can be remotely activated, deactivated, or activated for a pre-determined period of time. In certain embodiments, the sanitizing system 100 can provide two-way communication to the user, providing information regarding the sterilization process. Optionally, the sanitizing system 100 can be remotely operated by a smart phone device or other computing device.

The sanitizing system 100 can further include a display to reflect the operation of the sanitizing system 100. The display can display parameters regarding the lighting assemblies 120, including, but not limited to the emittance strength of the lighting assemblies 120, the duration of emittance, and/or time remaining on a timed emittance cycle. The display can further display parameters regarding the air filtration assembly, including, but not limited to the speed of the blower motor, the duration of air filtration, and/or time remaining on a timed filtration cycle. The display can be mounted on an external surface of the system housing 102. In some embodiments, the display can be mounted to allow the display to be viewed from outside the vehicle compartment.

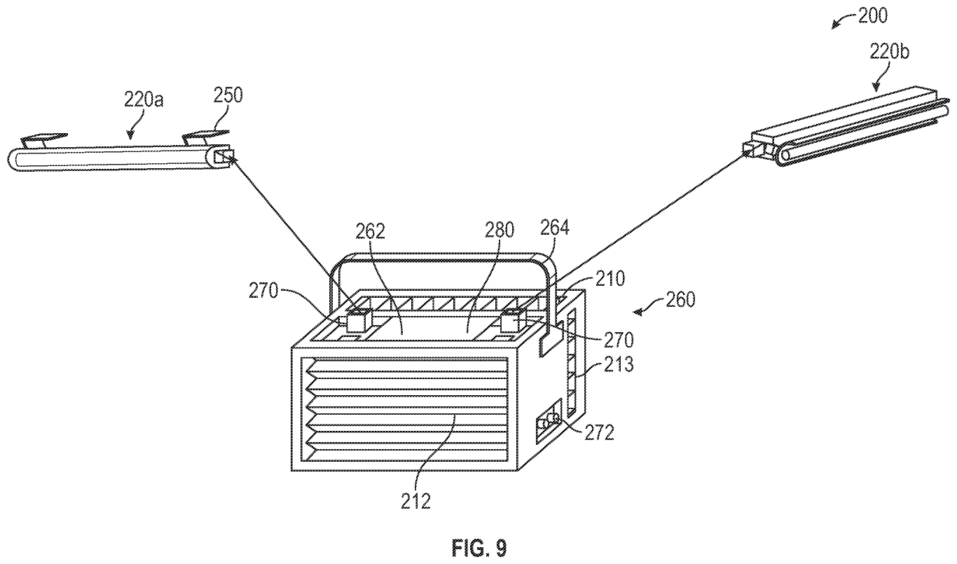

FIG. 9 is a perspective view of a representative system 200 in accordance with some embodiments. With reference to FIG. 9, the sanitizing system 200 can be introduced into a vehicle compartment to sanitize the vehicle compartment. Similar to sanitizing system 100, the sanitizing system 200 can sanitize the vehicle compartment by exposing surfaces within the vehicle compartment to radiation, such as UV light, to disinfect the vehicle compartment. Accordingly, sanitizing system 200 can include features that are similar to features of sanitizing system 100. Therefore, similar reference numerals may be utilized to refer to similar features.

In the depicted example, the sanitizing system 200 can be temporarily introduced into a vehicle compartment. As described herein, the service assembly 260 and the lighting assemblies 220a, 220b can be separate or modular components that are interconnected prior to service. In some applications, the sanitizing system 200 is a self-powered system, and may contain a power source, such as a battery to power the operation of the sanitizing system. In some embodiments, the sanitizing system 200 can connect to a vehicle to receive power from the vehicle for operations.

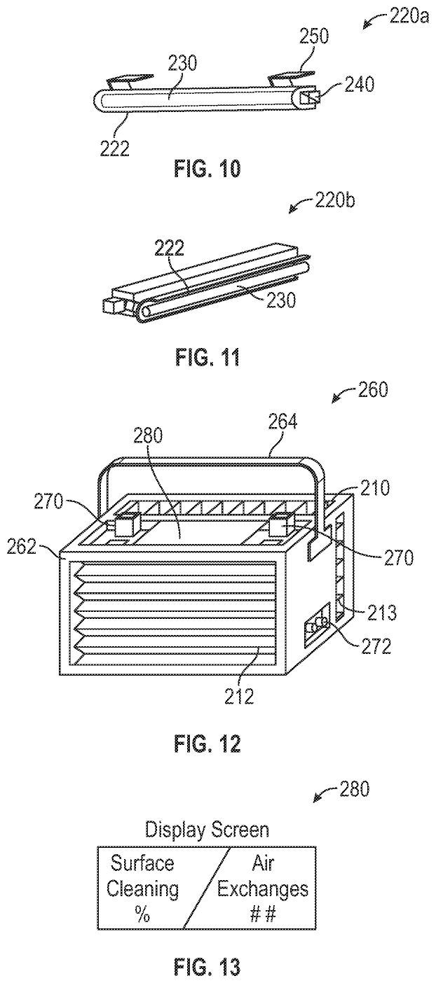

FIG. 10 is a perspective view of a representative lighting assembly 220a in accordance with some embodiments. FIG. 11 is a perspective view of a representative lighting assembly 220b in accordance with some embodiments. With reference to FIGS. 9-11, the sanitizing system 200 includes one or more modular lighting assemblies 220a, 220b, to direct radiation, such as UV light, to surfaces within the vehicle compartment to sanitize the vehicle compartment. As can be appreciated, while two lighting assemblies 220a, 220b, are shown, the sanitizing system 200 can include any suitable number of light assemblies in accordance with the vehicle compartment to be sanitized.

As illustrated, lighting assemblies 220a, 220b are separate from the service assembly 260, allowing the light assemblies to be disposed at any desired position within the vehicle compartment. The lighting assemblies 220a, 220b may be removably attached to the service assembly 260 for transport and/or storage. In some embodiments, the lighting assemblies 220a, 220b can be controlled and/or powered by the service assembly 260 via cables or wires therebetween.

Similar to lighting assembly 120, the lighting assemblies 220a, 220b can include a bulb or light source 230 that radiates UV light and a reflector 222 to direct the light emitted from the light source 230. As can be appreciated, the light source 230 can include similar features as light source 130 and the reflector 222 can include similar features as reflector 222. In some embodiments, such as lighting assembly 220b of FIG. 11, the lighting assembly 220b may not include an actuator to move the reflector 222.

With reference to FIG. 10, the lighting assembly 220a can be mounted to various portions of the vehicle compartment. The lighting assembly 220a can include a mounting mechanism 250 that allows the lighting assembly 220a to be attached to features of the vehicle compartment, including but not limited to windows, doors, visors, shelves, etc. In some embodiments, the mounting mechanism 250 includes a spring or biasing mechanism to allow a friction fit with various features of the vehicle compartment. Optionally, the mounting mechanism 250 can engage with features of the vehicle compartment, or can be secured within the vehicle compartment (e. g. secured in between a window and window frame, or between a door and a door frame).

With reference to FIG. 11, the lighting assembly 220b can be placed in various portions of the vehicle compartment. The lighting assembly 220b can be placed on the dashboard, on seats, on floorboards, etc. of the vehicle compartment. As can be appreciated, the sanitizing system 200 can utilize any number and combination of lighting assemblies that can be mounted and/or placed within the vehicle compartment. In various applications, the light assemblies 220a, 220b can be mounted in a variety of locations while allowing for desired levels of exposure or dosing of UV-C light. Optionally, the lighting assemblies 220a, 220b can be mounted or positioned in high positions (e. g. ceiling mount), low positions (e. g. floor mount), or other positions (along or on doors or windows, etc.).

FIG. 12 is a perspective view of a representative service assembly 260 in accordance with some embodiments. With reference to FIG. 12, the service assembly 260 provides power to the lighting assemblies 220a, 220b. The service assembly housing 262 can house a power source, such as a battery to power the operation of the lighting assemblies 220a, 220b. In some embodiments, the service assembly 260 can connect to a vehicle with terminals 272 to provide power to the lighting assemblies 220a, 220b. Additional functionality of the sanitizing system 200 can also be powered by the power source or the vehicle.

In the depicted example, the service assembly 260 can transfer power to the lighting assemblies 220a, 220b via the connectors 270. Cables or wires extending from the lighting assemblies 220a, 220b can be plugged or unplugged from the service assembly 260, allowing for service assembly 260 to be separated from the lighting assemblies 220a, 220b.

As illustrated, the service assembly 260 can be a portable device that is introduced into the vehicle compartment. The service assembly 260 can include a handle 264 extending from the service assembly housing 262 to facilitate handling and transport of the service assembly 260. Advantageously, the modular construction of the sanitizing system 200 allows the service assembly 260 to be placed in a convenient location while allowing the lighting assemblies 220a, 220b to be placed in desired or effective locations for sterilization.

Optionally, the service assembly 260 house an air filtration assembly to filter, purify, or exchange air within the vehicle compartment. Similar to sanitizing system 100, the air filtration assembly can filter air drawn in from the air inlet 210 and directed into the vehicle compartment through the air outlet 212. In some applications, the features of the air filtration assembly of the service assembly 260 are similar to the air filtration assembly of sanitizing system 100.

The air inlet 210 can be formed through a portion of the service assembly housing 262. As illustrated, the air inlet 210 can be positioned on an upper surface of the service assembly housing 262. The air inlet 210 can have a generally rectangular shape and may extend along the length of the service assembly housing 262. Optionally, the air inlet 210 can be disposed adjacent to the connectors 270. As can be appreciated, the air inlets 210 can be direct fluid communication with the vehicle compartment of the vehicle. In some embodiments, the air inlets 210 can be coupled to ducting to draw in air from the environment surrounding the vehicle to allow fresh airflow to enter the vehicle compartment.

After filtration, the air can be directed into the vehicle compartment through one or more air outlets 212. The service assembly 260 can further include a secondary air outlet 213. The air outlets 212, 213 can be formed through a portion of the service assembly housing 262. The air outlets 212, 213 can have a generally rectangular shape and may extend along the length of the service assembly housing 262. The air outlet 212 may be positioned on a face of the service assembly housing 262. The air outlet 213 may be positioned on a portion of a side face of the service assembly housing 262. The air outlet 213 may be positioned adjacent to the terminals 272. As can be appreciated, the air outlets 212, 213 can be direct fluid communication with the vehicle compartment of the vehicle. In some embodiments, the air outlets 212, 213 can be coupled to ducting to direct air from the service assembly 260 into the environment, expelling air from within the vehicle compartment.

In some embodiments, the service assembly 260 can include an air ionizer, air conditioning components, and/or heating elements within the service assembly housing 262. The air ionizer, air conditioning components, and/or heating elements can include features as described with respect to sanitizing system 100.

In the depicted example, the service assembly 260 can include a controller within the service assembly housing 262. As described with respect to sanitizing system 100, the controller can control the operation and power to the lighting assemblies 220a, 220b, the air filtration assembly, the air ionizer, the air conditioning components, and/or the heating elements of the sanitizing system 200.

FIG. 13 is a perspective view of a representative display 280 in accordance with some embodiments. With reference to FIGS. 12 and 13, the service assembly 260 can include a display 280 to reflect the operation of the sanitizing system 200. Similar to the display described with respect to sanitizing system 100, the display 280 can display parameters regarding the lighting assemblies 220a-220b, including, but not limited to the emittance strength of the lighting assemblies 220a-220b, the duration of emittance, and/or time remaining on a timed emittance cycle. The display 280 can further display parameters regarding the air filtration assembly, including, but not limited to the speed of the blower motor, the duration of air filtration, and/or time remaining on a timed filtration cycle. Optionally, the display 280 can display the state-of-charge of the battery within the service assembly housing 262. As illustrated, the display 280 can be mounted on an external surface of the service assembly housing 262. In some embodiments, the display 280 can be mounted to allow the display to be viewed from outside the vehicle compartment.

It will also be understood that, although the terms first, second, etc. are, in some instances, used herein to describe various elements, these elements should not be limited by these terms. These terms are only used to distinguish one element from another. For example, a first valve could be termed a second valve, and, similarly, a second valve could be termed a first valve, without departing from the scope of the various described embodiments. The first valve and the second valve are both valves, but they are not the same valve unless explicitly stated.

The terminology used in the description of the various described embodiments herein is for the purpose of describing particular embodiments only and is not intended to be limiting. As used in the description of the various described embodiments and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” is, optionally, construed to mean “when” or “upon” or “in response to determining” or “in response to detecting” or “in accordance with a determination that,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]” or “in accordance with a determination that [a stated condition or event] is detected,” depending on the context.

The foregoing description, for purpose of explanation, has been described with reference to specific embodiments. However, the illustrative discussions above are not intended to be exhaustive or to limit the scope of the claims to the precise forms disclosed. Many modifications and variations are possible in view of the above teachings. The embodiments were chosen in order to best explain the principles underlying the claims and their practical applications, to thereby enable others skilled in the art to best use the embodiments with various modifications as are suited to the particular uses contemplated.

What is claimed is:

1. A sanitizing system for sanitizing a compartment of a vehicle, the sanitizing system comprising:
a sanitizing system housing configured to be disposed within the compartment of the vehicle;
an air filtration assembly disposed at least partially within the housing, said air filtration assembly comprising:
an air inlet configured to be disposed in fluid communication with a compartment of a vehicle;
an air outlet configured to be in fluid communication with the compartment of the vehicle;
an air filter disposed between the air inlet and the air outlet; and
a blower fan configured to direct air from the air inlet to the air outlet through the filter; and
an ultraviolet (UV) lighting assembly comprising:
a UV light source fitting configured to receive a UV light source; and
a reflector disposed between the UV light source and a portion of the sanitizing system housing, the reflector configured to direct UV light from the UV light source into the compartment of the vehicle.

2. The sanitizing system of claim 1, wherein the sanitizing system housing is configured to be permanently coupled to the compartment of the vehicle.

3. The sanitizing system of claim 2, wherein the sanitizing system housing is configured to be permanently coupled to a ceiling of the compartment of the vehicle.

4. The sanitizing system of claim 2, wherein the sanitizing system housing is configured to be permanently coupled to a floor of the compartment of the vehicle.

5. The sanitizing system of claim 1, wherein the air outlet is configured to direct airflow through the UV light emitted by the UV light source.

6. The sanitizing system of claim 1, wherein the reflector comprises semi-circular cross-sectional profile.

7. The sanitizing system of claim 6, wherein the reflector comprises a tab to permit an operator to rotate the reflector.

8. The sanitizing system of claim 1, further comprising a controller configured to:
control emittance of UV light emitted by the UV lighting assembly within the compartment of the vehicle; and
filter air by the air filtration assembly within the compartment of the vehicle.

9. The sanitizing system of claim 8, wherein the controller is configured to simultaneously control emittance of UV light and filter air by the air filtration assembly.

10. The sanitizing system of claim 8, the lighting assembly further comprising an actuator coupled to the reflector, wherein the actuator is configured to rotate the reflector, and the controller is further configured to:
control rotation of the reflector to control dosing of ultraviolet light emitted by the lighting assembly within the compartment of the vehicle.

11. The sanitizing system of claim 8, further comprising a remote control wirelessly coupled to the controller, wherein the remote control is configured to remotely activate and deactivate the sanitizing system.

12. The sanitizing system of claim 8, further comprising a display operatively coupled to the controller, wherein the display is configured to:
display an operation metric of the lighting assembly within the compartment of the vehicle; and
display an exchange of air by the air filtration assembly within the compartment of the vehicle.

13. The sanitizing system of claim 8, further comprising a timer configured to start the system and turn off the system after a predetermined time set by an operator.

14. The sanitizing system of claim 8, wherein the controller is operatively coupled to the vehicle.

15. The sanitizing system of claim 14, wherein the controller is further configured to:
control operation of the sanitizing system when the vehicle is in an engine off state.

16. The sanitizing system of claim 14, wherein the controller is further configured to:
control operation of the sanitizing system when the vehicle is unoccupied.

17. The sanitizing system of claim 1, further comprising an air ionizer in fluid communication with the air inlet and the air outlet.

18. The sanitizing system of claim 1, further comprising a heating element.

19. A sanitizing system for sanitizing a compartment of a vehicle, the sanitizing system comprising:
a service assembly comprising:
a service housing configured to be disposed within the compartment of the vehicle;
an air inlet defined in the service housing, the air inlet in fluid communication with the compartment of the vehicle;
an air outlet defined in the service housing, the air outlet in fluid communication with the compartment of the vehicle;
a filter disposed between the air inlet and the air outlet; and
a blower disposed within the service housing and configured to direct air from the air inlet to the air outlet through the filter; and
at least one lighting assembly operatively coupled to the service assembly, the at least one lighting assembly comprising:
a lighting assembly housing;
a light source coupled to the lighting assembly housing, the light source configured to emit ultraviolet light when energized; and
a reflector disposed between the light source and the lighting assembly housing, the reflector defining a beam angle to direct the ultraviolet light into the compartment of the vehicle.

20. A method to sanitize a compartment of a vehicle, the method comprising:
directing air from within the compartment of the vehicle through a filter via an air inlet;
reintroducing the air into the compartment of the vehicle after passing through the filter via an air outlet;
dosing the compartment of the vehicle with ultraviolet light via a light source;
directing the ultraviolet light within the compartment of the vehicle in a beam angle direction via a reflector; and
rotating the reflector relative to the light source to control dosing of the compartment of the vehicle with ultraviolet light.
